SEQUENCE LISTING (3 sequences)

SEQ ID NO: 1 - DNA, 15 bp, Artificial
  Synthetic Primer V3fwd
tacggraggcagcag

SEQ ID NO: 2 - DNA, 23 bp, Artificial
  Synthetic Primer V4rev
atcttaccagggtatctaatcct

SEQ ID NO: 3 - DNA, 18 bp, Artificial
  Synthetic Primers V3fwd and X926_Rev
ccgtcaattcmtttragt

(19) United States
(12) Patent Application Publication
(10) Pub. No.: US 2021/0395343 A1
(43) Pub. Date: Dec. 23, 2021

(54) IVIG COMPOSITION AND METHOD OF TREATMENT OF ANTIBODY DEFICIENT PATIENTS

(71) Applicants: INSERM (INSTITUT NATIONAL DE LA SANTÉ ET DE LA RECHERCHE MÉDICALE), Paris (FR); SORBONNE UNIVERSITÉ, Paris (FR); ASSISTANCE PUBLIQUE-HÔPITAUX DE PARIS (APHP), Paris (FR)

(72) Inventors: Guy GOROCHOV, Paris (FR); Martin LARSEN, Paris (FR); Delphine STERLIN, Paris (FR); Jehane FADLALLAH, Paris (FR)

(21) Appl. No.: 17/287,727

(22) PCT Filed: Oct. 28, 2019
(86) PCT No.: PCT/EP2019/079369

§ 371 Date: Apr. 22, 2021

(30) Foreign Application Priority Data
Oct. 29, 2018 (EP) 18306409.6

(51) Int. Cl. C07K 16/06; A61P 37/02

(52) U.S. Cl. CPC C07K 16/06; A61P 37/02

(57) ABSTRACT

The invention is in the field of therapy of antibody deficiencies such as immune diseases and inflammatory disorders. The inventors demonstrate for the first time the convergence of intestinal IgA and serum IgG responses toward the same microbial targets, under homeostatic conditions. Private anti-microbiota IgG specificities are induced in IgA-deficient patients, but are not found in IgG pools from healthy donors, partially explaining why substitutive IgG (IVIG) cannot regulate antibody deficiency-associated gut dysbiosis and intestinal translocation. Finally, in both controls and IgA-deficient patients, systemic anti-microbiota IgG responses correlate with reduced inflammation suggesting that systemic IgG responses contribute to the gut microbiota confinement. Accordingly, the invention relates to IVIGs (Intravenous immunoglobulins) composition containing at least 1% of immunoglobulins (Ig) from SIgAd (Selective IgA deficiency) patient and their use in the treatment of antibody deficiency disorders such as immune diseases, inflammatory disorders and autoimmune disease.

### FIELD OF THE INVENTION

The invention is in the field of therapy of antibody deficiencies such as immune diseases and inflammatory disorders. In particular, the invention relates to IVIGs (Intravenous immunoglobulins) composition containing at least 1% of immunoglobulins G (IgG) from SIgAd (Selective IgA deficiency) patient and their use in the treatment of antibody deficiency disorders such as immune diseases (especially common variable immunodeficiency (CVID)) and inflammatory disorders (especially gut inflammatory diseases) and autoimmune disorders (especially in neurology, nephrology, rheumatology and dermatology).

### BACKGROUND OF THE INVENTION

Gut commensal bacteria contribute to several beneficial properties to the host. This complex community provides metabolic functions, prevents pathogen colonization and enhances immune development. A symbiotic relationship is maintained using host innate and adaptive immune responses such as antimicrobial compounds and mucus secretion, as well as IgA production^{1,2}. However, the gastrointestinal tract remains an important reservoir for potential bloodstream infections that involve Enterobacteriaceae, Enterococcus species or other Gram-negative bacilli^{3,4}. The physical gut barrier, but also innate and adaptive immune mechanisms, control host-microbiota mutualism, reducing the risk of bacterial translocation and systemic immune activation. Murine models of innate immune deficiency develop high seric IgG levels against gut microbiota². Significant titers of IgG targeting E. coli were also reported either in patients with inflammatory bowel diseases or in mice lacking secretory IgA^{5,6}. Nevertheless, based on recent murine studies, the notion has emerged that induction of systemic IgG responses against gut symbiotic bacteria is not necessarily a consequence of mucosal immune dysfunction or epithelial barrier leakiness. Healthy mice actively generate systemic IgG against a wide range of commensal bacteria under homeostatic conditions, which are passively transferred to the neonates through the maternal milk⁷. Serum IgG that specifically recognize symbiotic Gram-negative bacteria confer protection against systemic infections by these same bacteria. Because such IgG target a conserved antigen in commensal and pathogens, they also enhance elimination of pathogens such as Salmonella⁸.

IgG-expressing B cells are present in human gut lamina propria during steady state conditions, and represent 3-4% of the total gut B cells. About two-third of IgG+ lamina propria antibodies react with common intestinal microbes ⁹. Inflammatory bowel disease is associated with a marked increase in gut IgG⁺ B cells that might contribute to the observed elevated serum anti-E. coli IgG levels in these patients⁹. However, to which extent gut IgG⁺ B cells contribute to the serum IgG repertoire, remains elusive. Focusing on anti-transglutaminase 2 antibodies, it has been shown a low degree of clonal relationship between serum and intestinal IgG¹⁰. Altogether, it remains unknown whether secretory and serum anti-bacteria antibodies have identical targets or whether digestive and systemic antibody repertoires are shaped by distinct microbial consortia.

### SUMMARY OF THE INVENTION

The invention is based on the discovery that human serum IgG bind a broad range of commensal bacteria. Inventors also demonstrate for the first time the convergence of intestinal IgA and serum IgG responses toward the same microbial targets, under homeostatic conditions. Private anti-microbiota IgG specificities are induced in IgA-deficient patients, but are not found in IgG pools from healthy donors, partially explaining why substitutive IgG (IVIG) cannot regulate antibody deficiency-associated gut dysbiosis and intestinal translocation. Finally, in both controls and IgA-deficient patients, systemic anti-microbiota IgG responses correlate with reduced inflammation suggesting that systemic IgG responses contribute to the gut microbiota confinement.

Thus, the invention relates a composition of IVIGs (Intravenous immunoglobulins) containing at least 1% of immunoglobulin G (IgG) from SIgAd (Selective IgA deficiency) patient.

A further object of the invention relates to a therapeutic composition comprising composition of IVIG as defined above for the treatment of antibody deficiency disorders such as immune diseases (especially common variable immunodeficiency (CVID)), and inflammatory disorders especially gut inflammatory diseases and autoimmune disorders (neurology, nephrology, rheumatology and dermatology fields).

### DETAILED DESCRIPTION OF THE INVENTION

As previously mentioned the inventors demonstrate that serum anti-microbiota IgG are present in healthy individuals, and increased in SIgAd patients. IgG converge with non-overlapping secretory IgA repertoires to target the same bacteria. Each individual targets a diverse, microbiota repertoire whose proportion inversely correlates with systemic inflammation. Finally, actual Intravenous Immunoglobulin (IVIG) preparations target much less efficiently CVID (common variable immunodeficiency) gut microbiota than healthy microbiota. These data also suggest that IVIG preparations might be supplemented with IgG from IgA deficient patient's pools in order to offer a better protection against gut bacterial translocations in CVID.

### IVIG Preparation

Based on this knowledge, the inventors propose a composition of IVIGs (Intravenous immunoglobulins), which could be used in order to treat antibody deficiency disorders.

Thus, the invention relates to a composition of IVIGs (Intravenous immunoglobulins) containing at least 1% of immunoglobulin G (IgG) from SIgAd (Selective IgA deficiency) patients.

In a specific embodiment, the composition of IVIGs according to the invention contain at least 1%, 10%, 20%, 30%, 40%, 50%, 60%, 70%, 80%, 90%, 95%, 98 or 99% of immunoglobulin G (IgG) from SIgAd (Selective IgA deficiency) patients.

In a specific embodiment, the composition of IVIGs according to the invention contain 100% of immunoglobulin G (IgG) from SIgAd (Selective IgA deficiency) patients.

In a specific embodiment, the composition of IVIGs according to the invention, contain between 1% to 10% of immunoglobulin G (IgG) from SIgAd (Selective IgA deficiency) patients.

In a particular embodiment, the composition of IVIGs according to the invention, contain 1%, 2%, 3%, 4%, 5%, 6%, 7%, 8%, 9% or 10%, of immunoglobulin G (IgG) from SIgAd (Selective IgA deficiency) patients.

The term “IVIG” means “Intravenous immunoglobulin” a blood product prepared from the serum of between 1000 and 15000 donors per batch. The active substances in IVIG preparations are polyclonal natural antibodies synthesized, in response to immune stimuli (antigens and T cells), by plasma B cells. Intravenous immunoglobulins (IVIGs) are a therapeutic preparation of pooled normal polyspecific human IgGs obtained from large numbers of healthy donors. The preparation contains antibodies to microbial antigens, self antigens (natural autoantibodies) and anti-idiotypic antibodies which recognize other antibodies [Durandy, A. et al. Clin. Exp. Immunol. 2009, 158, 2-13]. These categories are not mutually exclusive.

IVIG is the treatment of choice for patients with antibody deficiencies. For this indication, IVIG is used at a ‘replacement dose’ of 400-600 mg/kg body weight, given approximately 3-weekly. In contrast, ‘high dose’ IVIG (hdIVIG), given most frequently at 2 g/kg/month, is used as an ‘immunomodulatory’ agent in an increasing number of immune and inflammatory disorders.

Methods for obtaining/producing such “Intravenous immunoglobulin” are well known in the art. Examples of methods for obtaining/producing IVIGs include but are not limited to any methods described in Afonso A. et al (Biomolecules 2016, 6, 15); all of which are herein incorporated by reference

Plasma used in the production of IVIG comes from two origins: approximately 20 percent is from blood donors, and the other 80 percent is from plasma donors. Individual plasmas are pooled; the pool size is a minimum of 1000 donors, but may be up to 100,000 donors (Radosevich, M.;et al Vox Sang. 2010, 98, 12-28). The maximum number of donors in pools is treated as proprietary information by each manufacturer. The many thousands of donors who contribute to a typical pool of plasma used for isolation of immunoglobulin represent a wide range of antibody specificities against infectious agents (Looney, R. Jet al Best Pract. Res. Clin. Haematol. 2006,19, 3-25.,15 and European Medicines Agency Guideline on the Clinical Investigation Of Human Normal Immunoglobulin For Intravenous Administration (IVIg). Available online: www.ema.europa.eu/docs/en_GB/document library/Scientific guideline/2009/10/WC500004 766.pdf) such as bacterial, viral and also a large number of self antigens reflecting the cumulative exposure of the donor population to the environment.

Briefly, the main technique for industrial preparation of IVIGs are:
1) Fractionation: Techniques developed by Cohn (Cohn, E. J.; et al. J. Am. Chem. Soc. 1946, 68, 459-475.) based on the separation of plasma proteins into individual stable fractions with different biological functions. The basis for Cohn's fractionation was to use low concentrations of alcohol, reducing the pH and lowering ionic strength. The procedure was performed at low temperature, which reduced the likelihood of contamination and made large-scale fractionation possible. This method, further refined in cooperation with J. L. Oncley (Oncley, J. L.; et al J. Am. Chem. Soc. 1949, 71, 541-550.), is basically still in use and, with some additional steps, yields Ig for intravenous and subcutaneous use [Eibl, M. M. Immunol. Allergy Clin. North Am. 2008, 28, 737-764.).
2) Chromatography. Purification of immunoglobulins by ion-exchange chromatography on diethylaminoethyl (DEAE) cellulose columns was first reported by Fahey, J. L. et al. (J. Biol. Chem. 1959, 234, 2645-2651). DEAE chemical groups bear a positive charge and bind to ions (anions) and proteins that have an overall negative charge. Latter, IgG was separated from human serum in a 2-step batch procedure using DEAE-Sephadex (Baumstark, J. S.; et al . Arch. Biochem. Biophys. 1964, 108, 514-522.). 97% pure IgG could be recovered in the supernatant. Although the new generation of resins has an improved binding capacity (Staby, A.; et al. J. Chromatogr. A 2004, 1034, 85-97) large columns are still needed. Large buffer volumes have to be applied to completely elute the IgG from the column. The IgG fraction is highly diluted, resulting in large volumes. Hydrophobic Charge Induction Chromatography (HCIC) for the purification of antibodies was first described by Burton and Harding [Burton, S. C.; et al. J. Chromatogr. A 1998, 814, 71-81). The technique is based on the pH dependent behaviour of an ionisable dual mode ligand. Size exclusion chromatography is suited for the final phase of the separation process and allows the separation of the different IgG forms according to their respective sizes. Thus IgG solutions can be separated in poly-, di- and monomers under mild conditions. IVIg preparations prepared from pooled plasma of thousands of healthy donors contain monomeric and dimeric IgG, whereas IVIg isolated from one donor contains only IgG monomers

As previously described, the invention relates a composition of IVIGs (Intravenous immunoglobulins) containing at least 1% of immunoglobulin G (IgG), from SIgAd (Selective IgA deficiency) patients. For the preparation of the composition of IVIGs according to the invention the same technique for industrial preparation of IVIGs can be used.

Thus, the invention also relates to a method of preparation of the composition of IVIGs according to the invention by Fractionation and/or Chromatography technique.

The term “SIgAd” means “Selective IgA deficiency”. SIgAD is characterized by serum IgA level inferior of 0.07g/l and a concomitant lack of secretory IgA. SIgAd is the most common form of primary immunodeficiency (PID) in the western world and affects approximately 1/600 individuals in 2000's (Clin Exp Immunol 1997; 159:6236 41.). However, there is a marked variability in the prevalence in different ethnic groups (Hammarstrom L et al. Primary immunodeficiency diseases, a molecular and genetic approach. Oxford: Oxford University Press, 1999:250 62.), with a lower frequency in Japanese (1/18 000) and Chinese (1/4000). The term ‘selective IgAD’ should be reserved for those individuals who do not have identifiable disorders which are known to be associated with low IgA levels. However, in many cases a simultaneous change in the IgG subclass pattern is seen with a lack of specific anti-polysaccharide antibodies of the IgG2 subclass (Hammarström L, et al.. Immunology 1985; 54:821 6) or a total lack of serum IgG2 (Oxelius Vet al. N Engl J Med 1981; 304:1476 7.), IgG4 and IgE (Hammarström L, et al Monogr Allergy 1986; 20:234 5.), reflecting a relative or absolute block in switching to genes downstream of the G1.

### Pharmaceutical Composition

The composition of IVIGs of the present invention, together with one or more conventional adjuvants, carriers, or diluents may be placed into the form of pharmaceutical compositions and unit dosages.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The pharmaceutical compositions and unit dosage forms may comprise conventional ingredients in conventional proportions, with or without additional active compounds or principles, and the unit dosage forms may contain any suitable effective amount of the active ingredients commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal administration; or in the form of sterile injectable solutions for parenteral uses. Formulations containing about one (1) milligram of active ingredient or, more broadly, about 0.01 to about one hundred (100) milligrams, per tablet, are accordingly suitable representative unit dosage forms.

The composition of IVIGs of the present invention is formulated for parenteral administration (e.g., by injection, for example bolus injection or continuous infusion) and may be presented in unit dose form in ampoules, pre-filled syringes small volume infusion or in multi-dose containers with an added preservative. The compositions may take such forms as suspensions, solutions, or emulsions in oily or aqueous vehicles, for example solutions in aqueous polyethylene glycol. Examples of oily or non-aqueous carriers, diluents solvents or vehicles include propylene glycol, polyethylene glycol, vegetable oils (e.g., olive oil, and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

### Method of Preventing or Treating Antibody Deficiency Disorders

Another object of the invention is a method for treating antibody deficiency disorders more particularly inherited or acquired immunodeficiencies such as in immune diseases, inflammatory disorders, and autoimmune disorders, comprising administering to a subject in need thereof a therapeutically effective amount of a composition of IVIGs according to the invention as disclosed above.

In one embodiment, the immune disease is Primary antibody deficiency (PAD) or secondary antibody deficiencies (SAD).

As used herein, the term “primary antibody deficiencies” has its general meaning in the art and refers to a group of rare disorders characterized by an inability to produce clinically effective immunoglobulin responses. Example of primary antibody deficiencies that may be treated by methods and composition of the invention include Bruton's disease β-cell intrinsic, Good's syndrome, Hyper IgM Syndrome (HIGM), Wiskott-Aldrich syndrome (WAS) X-linked agammaglobulinemia (XLA), common variable immunodeficiency (CVID), selective IgA deficiency, specific antibody deficiency and transient hypogammaglobulinaemia of infancy (THI).

In one embodiment, the PAD is common variable immunodeficiency (CVID).

As used herein, the term “secondary antibody deficiencies” has its general meaning in the art and are defined by a quantitative or qualitative decrease in antibodies that occur most commonly as a consequence of renal or gastrointestinal immunoglobulin loss, hematological malignancies and corticosteroid, immunosuppressive or anticonvulsant medications.

In one embodiment the secondary antibody deficiencies is selected from the list consisting of myeloma, chronic lymphocytic leukemia (CLL), and immune deficiencies induced by treatment (immunosuppressive or cytostatic drugs).

In one embodiment, the inflammatory disorders is gut inflammatory such as inflammatory bowel diseases, sepsis or graft versus host disease.

Accordingly, the composition of IVIGs according to the invention is used for the treatment of antibody deficiency disorders selected form the list consisting of immune diseases, inflammatory disorders and autoimmune disease.

By a “therapeutically effective amount” is meant a sufficient amount of compound to treat and/or to prevent antibody deficiency disorders such as immune diseases especially common variable immunodeficiency (CVID), and inflammatory disorders especially gut inflammatory diseases (Inflammatory Bowel Diseases).

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved.

The composition of IVIGs according to the invention is administered by parenteral (including intramuscular, intra-arterial, intrathecal, subcutaneous and intravenous).

The term “antibody deficiency disorders” means diseases caused directly or indirectly by immunodeficiency like in immune diseases such as Primary antibody deficiency (PAD) (especially common variable immunodeficiency (CVID)) and Secondary antibody deficiencies (SAD). Antibody deficiency disorders are frequently associated with inflammatory disorders (especially gut inflammatory diseases but also sepsis, graft versus host disease) and autoimmune disease. Antibody deficiency disorders (Immune disease, Inflammatory disorders and autoimmune disease) affect the neurological, haematological, nephrological, rheumatological and dermatological spheres.

The composition of IVIGs according to the invention (supplemented with SIgAd serum patient) can also be used for Ig replacement therapy in all IVIG indications currently accepted.

The clinical specialities using the largest amounts of IVIG are presently haematology and immunology (for supplementation of Ig deficiency and also for autoimmune disease) and neurology, nephrology, rheumatology and dermatology (for autoimmune disease).

In autoimmune disease IVIG has had a major impact on the treatment of neurological disorders including dermatomyositis, Guillain—Barre syndrome, chronic inflammatory demyelinating polyneuropathy (CIDP), multifocal motor neuropathy (MMN), myasthenia gravis and stiff person syndrome. For autoimmune disease in nephrology, rheumatology and ophthalmology it has been used to treat vasculitis, systemic lupus erythematosis (SLE), mucous membrane pemphigoid and uveitis and in dermatology it is used most commonly to treat Kawasaki syndrome, dermatomyositis, toxic epidermal necrolysis and the blistering diseases (for review see Jolles S. et al Clinical and Experimental Immunology (2005) 142:1-11).

For immune disease in haematology it is used to treat immune cytopenias, parvovirus B19 associated red cell aplasia, hypogammaglobulinaemia secondary to myeloma and chronic lymphatic leukaemia and post-bone marrow transplantation. For immune disease in immunology, IVIG is used in the treatment of primary antibody deficiency (PAD: such as X-linked agammaglobulinemia (XLA), CVID, Hyper IgM Syndrome (HIGM), Wiskott—Aldrich syndrome (WAS) and Secondary antibody deficiencies (myeloma, Chronic Lymphocitic Leukemia, drugs and other causes).

The composition of IVIGs according to the invention can be specially used for the treatment of common variable immunodeficiency (CVID) and associated dysbiosis and/or gut bacterial translocations as currently used IVIG poorly target CVID microbiota.

The term “CVID” means “common variable immunodeficiency”. CVID is an immune disorder characterized by recurrent infections and low antibody levels, specifically in immunoglobulin (Ig) types IgG, IgM and IgA. Symptoms generally include high susceptibility to foreign invaders, chronic lung disease, inflammation and infection of the gastrointestinal tract. However, symptoms vary greatly between people. CVID is a lifelong disease. CVID affects about 1/25000 Caucasians, the patients having a marked reduction in serum levels of both IgG (usually <3 g/l) and IgA (<0.05 g/l); IgM is also reduced in about half the patients (<0.3 g/l) (Clin Exp Immunol 1997; 159:6236 41). Symptoms of recurring infection can start at any time of life, but there are peaks of onset during 1-5 and 16-20 years of age (Hermaszewski RA et al Quart J Med 1993; 86:31 42), with equal distribution between the sexes. The condition is clinically more complex than X—linked agammaglobulinaemia (XLA), with patients being prone to chronic inflammatory and autoimmune complications (Cunningham—Rundles C et al. J Clin Immunol 1999; 92:34 48).

The term “Dysbiosis” also called “Dysbacteriosis” means a microbial imbalance on or inside the body, such as an impaired microbiota. For example, a part of the human microbiota, such as the skin flora, gut flora, or vaginal flora, can become deranged, with normally dominating species underrepresented and outcompeted by species increasing to fill the void. Dysbiosis is most commonly reported as a condition in the gastrointestinal tract,

The term “inflammatory diseases” according to the invention means especially gut inflammatory diseases (inflammatory bowel diseases) or sepsis or graft versus host disease.

The composition of IVIGs according to the invention can be also specially used for the treatment of gut inflammatory diseases such as Inflammatory Bowel Diseases (IBD) or Irritable Bowel Syndrome (IBS).

As used herein, the term “inflammatory bowel diseases (IBD)” is a group of inflammatory diseases of the colon and small intestine. The major types of IBD are Crohn's disease, ulcerative colitis Celiac disease, and pouchitis.

As used herein, the term “Irritable Bowel Syndrome (IBS)” is a term for a variety of pathological conditions causing discomfort in the gastro-intestinal tract. It is a functional bowel disorder characterized by chronic abdominal pain, discomfort, bloating, and alteration of bowel habits in the absence of any organic cause. It also includes some forms of food-related visceral hypersensitivity, such as Gluten hypersensitivity.

The invention will be further illustrated by the following figures and examples. However, these examples and figures should not be interpreted in any way as limiting the scope of the present invention.

### FIGURES

FIG. 1: Systemic IgG and secretory IgA recognize a common spectrum of commensals. A. Representative flow cytometry dot plot showing from bottom to top isotype control, endogenous secretory IgA (without serum), human IgG anti-TNF (10 μg/ml; irrelevant IgG) and autologous systemic IgG (10 μg/ml) to fecal microbiota in a healthy donor. B. Flow cytometry analysis of the fraction of fecal microbiota bound by either secretory IgA, seric IgG or both in healthy donors (n=30). Median values are indicated and subgroups are compared with a non-parametric Mann-Whitney test.

FIG. 2: Systemic IgG bind a broad spectrum of commensals. A. Flow cytometry analysis of serum IgG binding to cultivated bacterial strains. Grey histograms represent isotype controls and dark lines anti-IgG staining. B. Flow cytometry analysis of serum IgG binding levels to 8 different bacterial strains in healthy donors (n=30). Blue strains (left) are typically poorly coated by secretory IgA from healthy individuals while pink strains (right) are representative of typical IgA targets¹⁵. Results are presented as A Median Fluorescence Intensity (MFI) i.e.: IgG=MFI IgG serum—MFI IgG negative control. Red bars show medians. Kruskal-wallis test was used to calculate p-value. C. Representative immunoblotting of Escherichia coli lysates probed with five different healthy human serums, with a normalized IgA and IgG levels. Ponceau staining indicates total amounts of bacteria lysates loaded. IgA and IgG binding were assessed by an HRP conjugated secondary antibody.

FIG. 3: IgA deficient patients harbour private anti-commensal IgG responses. A. Flow cytometry analysis of fecal microbiota bound by autologous seric IgG in healthy donors (n=30) and IgA deficient patients (n=15). Red bars represent medians. P-value was calculated by Mann-Whitney test. B. Representative flow cytometry analysis of autologous seric IgG binding (left) or polyclonal IgG derived from pooled serum of healthy donors binding (right) to fecal microbiota. In a healthy donor (top) and in an IgA deficient patient (bottom). C. Flow cytometry analysis of the IgG-bound fecal microbiota with IgG from autologous serum or polyvalent IgG in healthy donors (n=30) and IgA deficient patients (n=15). P-values were calculated by Wilcoxon-paired test. D. Flow cytometry detection of IgG on IgA deficient microbiota (n=9), following incubation with autologous serum or heterologous serum from another, randomly picked, IgA deficient individual. P-value was calculated by Wilcoxon-paired test.

FIG. 4: Private IgG anti-microbial signatures. A. Sorting strategy of IgG-bound and IgG-unbound microbiota in 10 healthy donors and 3 IgA deficient patients. Composition of sorted subsets was next analysed by 16S rRNA sequencing. B. Genera diversity in IgG+ and IgG− sorted fractions calculated by Shannon index. Dark symbols correspond to healthy donors, red symbols to IgA deficient patients. C. Median relative abundance of genera in IgG+ and IgG− sorted fractions. Dark symbols correspond to healthy donors, red symbols to IgA deficient patients.

FIG. 5: Microbiota specific IgG and inflammation A. Percentage of serum IgG-bound microbiota correlated with sCD14 levels in autologous serum of healthy donors (triangles) and SIgAd patients (dark points). Spearman coefficient (r) and p-value (p) are indicated. B. Flow cytometry analysis of IgG-bound microbiota following IVIG exposure in healthy donors and CVID patients. C. sCD14 levels measured by ELISA in plasmas of healthy donors and CVID patients. D. Seric IL-6 levels measured by Simoa technology in plasmas of healthy donors and CVID patients. E. Flow cytometry analysis of CD4+CD45RA-PD-1+ lymphocytes in peripheral blood mononuclear cells of healthy donors and CVID patients. Percentage among CD4+ T cells is presented. For all dot plots, black lines represent medians. Mann-Whitney test was used to calculate p-values (*p<0.05, ***p<0.001)

FIG. 6: In vivo intestinal IgG binding to gut microbiota. Flow cytometry analysis of the fraction of fecal microbiota bound by intestinal IgG in healthy donors (HD; n=30) and selective IgA deficient patients (SIgAd; n=15). Bars represent medians.

FIG. 7: Anti-commensals IgG react mostly in a Fab-dependent manner A-B. Flow cytometry analysis of 30 healthy (A) and 15 IgA deficient (B) fecal microbiota samples incubated with seric IgG or human IgG anti-TNF. C. Flow cytometry analysis of 10 IgA deficient fecal microbiota samples incubated with heterologous seric IgG or human IgG anti-TNF. Wilcoxon-paired test was used to calculate p-values. **p<0.01;***p<0.001; ****p<0.0001

### EXAMPLE 1

Material & Methods

Human Samples

Fresh stool and blood samples were simultaneously collected from n=30 healthy donors, n=15 selective IgA deficiency and n=10 common variable immunodeficiency patients.

Healthy donors were recruited among laboratory staff and relatives. Patients followed for clinical manifestations associated with antibody deficiencies were recruited from two French clinical immunology referral centers (Department of Clinical Immunology at Saint Louis hospital and Department of Internal Medecine at Pitrié-Salpêtrière hospital, Paris). Patient's inclusion criteria were (i) undetectable seric IgA levels (<0,07 mg/mL) in at least three previous samples in the past year (ii) either selective IgA deficiency (n=15 selective IgA deficient patients), or associated with IgG and/or IgM deficiency integrating a global antibody production defect (n=10 CVID patients). Clinical and biological data were collected at inclusion time.

Surgical samples from histologically normal intestine were obtained from twelve donors undergoing gastric bypass or tumorectomy at Pitié-Salpêtrière hospital, Paris.

Oral and written consent were obtained from patients and healthy donors before inclusion in the study.

PBMC and Plasma

30 mL of blood were collected in ACD tubes (BD Vacutainer®) and PBMC were isolated by density gradient procedure (Ficoll 400, Eurobio, Les Ulis, France) and then stored in liquid nitrogen after soft freezing in isopropanol. Supernatants were collected as plasma and immediately stored at −80° C.

Stool Collection and Whole Microbiota Purification

Stool were collected immediately after emission in a container allowing anaerobic bacteria preservation (Anaerocult band, Merck, Darmstadt, Germany), aliquoted in a CO2-rich 02-low atmosphere and stored at −80° C. Fecal microbiota were extracted by gradient purification in anaerobic conditions (Freter chamber) as previously described³⁷. Briefly, thawed feces were diluted in 1×-PBS (Eurobio), 0,03% w/v sodium deoxycholate (NaDC), 60% w/v Nycodenz (Sigma-aldrich, St Louis, USA) and loaded on a continuous density gradient obtained by a freezing-thawing cycle of a Nycodenz solution. Fecal bacteria were obtained after ultracentrifugation (14567×g, 45 min, +4° C.) (Beckman Coulter ultracentrifuge, swinging rotor SW28) and washed three times in 1×-PBS (Eurobio), 0,03% w/v sodium NaDC. The final pellet was diluted in 1×PBS-10%Glycerol, immediately frozen in liquid nitrogen and then stored at −80° C.

Bacterial Flow Cytometry

Specific seric antibodies levels against purified microbiota or cultivable strains were assessed by a flow cytometry assay as previously described¹¹. Briefly, 10⁷ bacteria (purified microbiota or cultivable strains) were fixed in a solution of 4% paraformaldehyde and simultaneously stained with a cell proliferation dye (eFluor 450, eBiosciences, Calif., USA). After washing with 1 mL of a 1×-PBS solution, cells were resuspended to a final concentration of 4.10⁸ bacteria/mL in a 1×-PBS, 2% w/v BSA, 0.02% w/v Sodium azide solution. Then 10⁷ bacteria were incubated in a 96-V bottom well plate with a 10 μg/mL IgG solution (from either human serum or pooled human IgG Hizentra®—CSL Behring France or human anti-TNF Remicade®—MSD France) per condition. Immune complexes were washed twice with a 1×-PBS, 2% w/v BSA, 0.02% w/v Sodium azide (200 μL/well, 4000×g, 10 minutes, +4° C.) and then incubated with secondary conjugated antibodies, either isotype controls mix or goat anti-human IgA-FITC and goat anti-human IgG-A647 (Jackson Immunoresearch Laboratories, West Grove, USA). Acquisition of the cells events was performed on a FACS CANTO II flow cytometer (Becton Dickinson) after washing and analysis was performed with Flow-Jo software (Treestar, Ashland, USA). Medians of fluorescence were used to measure the seric IgG response levels against the cultivable strains. Intestinal IgA binding was quantified by the same assay without incubation with seric immunoglobulins. Results are expressed as median, minimum and maximum percentages throughout the manuscript.

Cytokines Quantification

IL-6 and IL-10 were measured in the serum using a 3-step digital assay relying on Single Molecule Array (Simoa) technology HD-1 Analyzer (Quanterix Corporation, Lexington, USA). Working dilutions were ¼ for all sera in working volumes of 25 μL. Lower limit of quantification for IL-6 and IL-10 are respectively of 0.01, 0.021 pg/mL.

Soluble CD14 Quantification

Soluble CD14 was quantified in plasma (400-fold dilution) by ELISA (Quantikine® ELISA kit, R&D, Minneapolis, USA). Experimental procedure followed the manufacturer's recommendations. Lower limit of quantification for soluble CD14 is of 6 pg/mL.

Peripheral Blood Mononuclear Cell Phenotyping

T cell phenotyping was performed using a combination of the following antibodies : CD3-H500, CCR7-PE-Cy7, CD4-APC-Cy7 (BD Biosciences), CD45RA-PercP Cy5.5 (e-Bioscience), CD8-A405 (Invitrogen), CD279-APC (BioLegend). Acquisition of cells events was performed using a FACS CANTO II flow cytometer (Becton Dickinson) and analysis was performed using the Flow-Jo software (Treestar).

Intestinal B Cells Phenotyping

Lamina propria was digested by collagenase A (Roche) in RPMI (Life Technologies) for 30 minutes at 37° C. Lymphocytes were purified by centrifugation over Ficoll 400 (Eurobio) and stained with the following antibodies: anti-CD45 APC-H7, anti-CD19 BV421, anti-IgD FITC, anti-CD27 PE-Cy7 (all purchased from BD Biosciences), and anti-IgA PE (Jackson Immunoresearch), or anti-IgG1 PE, anti-IgG2 AF488, anti-IgG3 A647 (Southern Biotech). Dead cells were excluded with LIVE/DEAD™ Fixable Aqua Dead Cell Stain Kit (Invitrogen). Acquisition of cells events was performed using a FACS CANTO II flow cytometer (Becton Dickinson) and analysis was performed using the Flow-Jo software (Treestar).

Analysis of IgG-Coated Bacteria

Purified microbiota (10⁹/condition) was washed in 1×-PBS and stained with isotype control (A647-conjugated Goat IgG, Jackson Immunoresearch Laboratories) as a negative control or anti-human IgG-A647 (Jackson Immunoresearch Laboratories). Acquisition and sorting were performed on a 2 lasers-2 ways Fluorescent-activated cell sorter (S3 cell sorter, Bio-Rad Laboratories, California, USA). 10⁶ bacteria per fraction were collected and immediately stored at −80° C. as dry pellets. Purity for both fractions was systematically verified after sorting with a minimum rate of 80%. Genomic DNA was extracted and the V3-V4 region of the 16S rRNA gene was amplified by semi-nested PCR. Primers V3fwd (+357): 5′ TACGGRAGGCAGCAG 3′ (SEQ ID N° 1) and V4rev (+857): 5′ ATCTTACCAGGGTATCTAATCCT 3′ (SEQ ID N° 2) were used during the first round of PCR (10 cycles). Primers V3fwd and X926_Rev (+926) 5′ CCGTCAATTCMTTTRAGT 3′ (SEQ ID N° 3) were used in the second PCR round (40 cycles). Polymerase chain reaction amplicon libraries were sequenced using a MiSeq Illumina platform (Genotoul, Toulouse, France). The open source software package Quantitative Insights Into Microbial Ecology (QIIME)³⁸ was used to analysed sequences with the following criteria: (i) minimum and maximum read length of 250 bp and 500 bp respectively, (ii) no ambiguous base calls, (iii) no homopolymeric runs longer than 8 bp and (iv) minimum average Phred score >27 within a sliding window of 50 bp. Sequences were aligned with NAST against the GreenGenes reference core alignment set (available in QIIME as core_set_aligned.fasta.imputed) using the ‘align_seqs.py’ script in QIIME. Sequences that did not cover this region at a percent identity >75% were removed. Operational taxonomic units were picked at a threshold of 97% similarity using cd-hit from ‘pick_otus.py’ script in QUIIME. Picking workflow in QUIIME with the cd-hit clustering method currently involves collapsing identical reads using the longest sequence-first list removal algorithm, picking OTU and subsequently inflating the identical reads to recapture abundance information about the initial sequences. Singletons were removed, as only OTU that were present at the level of at least two reads in more than one sample were retained (9413±5253 sequences per sample). The most abundant member of each OTU was selected through the ‘pick_rep_set.py’ script as the representative sequence. The resulting OTU representative sequences were assigned to different taxonomic levels (from phylum to genus) using the GreenGenes database (release August 2012), with consensus annotation from the Ribosomal Database Project naïve Bayesian classifier [RDP 10 database, version 6³⁹. To confirm the annotation, OTU representative sequences were then searched against the RDP database, using the online program seqmatch (http://rdp.cme.msu.edu/segmatch/segmatch_intro.jsp) and a threshold setting of 90% to assign a genus to each sequence.

Immunoblotting

10⁸ CFU of wild type Escherichia coli were freezed (−80° C.) and thawed (37° C.) three times in 30 μL of lysis buffer (50mM Tris-HCL, 8M urea). Lysis efficiency was verified by Gram staining. Proteins were separated using 4%-20% polyacrylamide gel electrophoresis (Mini-PROTEAN TGX Stain-Free Precast Gels; Bio-Rad) in reducing conditions (dithiothreitol DTT and sodium dodecyl sulfate SDS, Bio-Rad) and transferred to nitrocellulose. Membranes were incubated with 10 μg/ml of human seric IgG or IgA of different healthy donors. Human IgG were detected with horseradish peroxidase-conjugated goat anti-human IgG used at 1:50,000 or goat anti-human IgG used at 1:20,000 followed by enhanced chemi-luminescence revealing reaction (Clarity™ Western ECL, Bio-Rad). Human IgA were detected with horseradish peroxidase-conjugated goat anti-human IgA used at 1:20 000 (Bethyl Laboratories). All incubations were in 1×-PBS with 5% non fat milk and washing steps in 1×-PBS with 0.1% Tween.

IgG Gene Expression Analysis

Total RNA of jejunal lamina propria fraction and PBMC were extracted with the RNeasy Mini kit (QIAGEN). cDNAs were synthesized from and prepared with M-MLV reverse transcriptase (Promega). SYBR green primers were designed by manufacturer (Roche) and used for qRT-PCR using the 7300 real time PCR system (Applied Biosystem). Data were normalized to ribosomal 18S RNA.

Results

1/Convergence of Intestinal IgA and Serum IgG Toward the Same Bacterial Cells

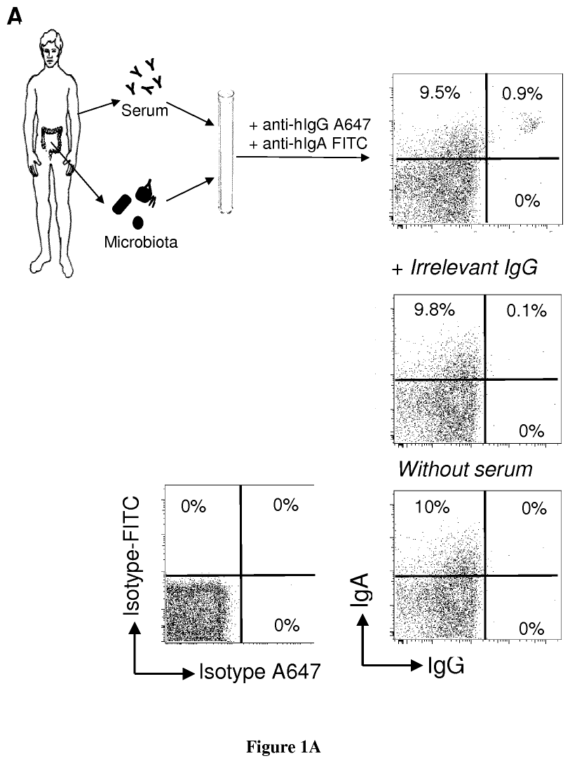

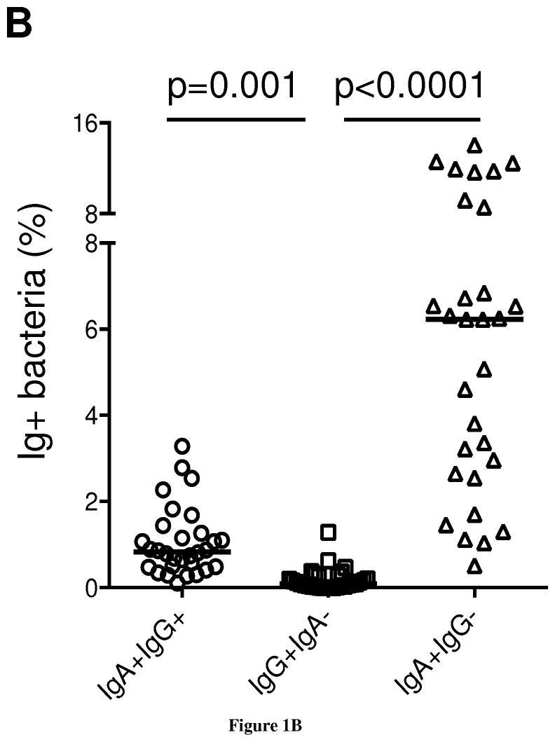

To determine the level of humoral systemic response against fecal microbiota, we have elaborated a flow cytometric assay derived from a previously reported technology¹¹. This protocol allows to probe concomitantly IgA and IgG microbiota coating. We found that approximately 8% of the fecal microbiota is targeted by secretory IgA (median[min-max]%; 8[0.8-26.7]%; n=30) in healthy donors, in concordance with previous reports¹². As shown, the proportion of bacteria in vivo bound by secretory IgA in human feces is highly variable between healthy individuals (FIG. 1B). IgG-bound bacteria are virtually absent from healthy human feces (median [min-max]%; 0.03[0-0.16]%; n=30 ; FIG. S1 and 1A), in agreement with the lack of IgG transport to the intestinal lumen. In healthy donors, seric IgG bound a median rate of 1.1% of fecal bacteria (median [min-max]%; 1.1[0.2-3.2]%; FIG. 1B). Surprisingly, seric IgG targeted exclusively secretory IgA bound bacteria (FIG. 1A). Conversely, all IgA-coated bacteria (IgA⁺ bacteria) were not targeted by seric IgG. Of note, an irrelevant human monoclonal IgG (chimeric anti-human TNF containing a human Fc IgG fraction) exhibits markedly reduced binding to IgA+bacteria, compared to serum IgG (FIG. 1A, S2), demonstrating that IgG binding to IgA-coated bacteria is mostly Fab-mediated.

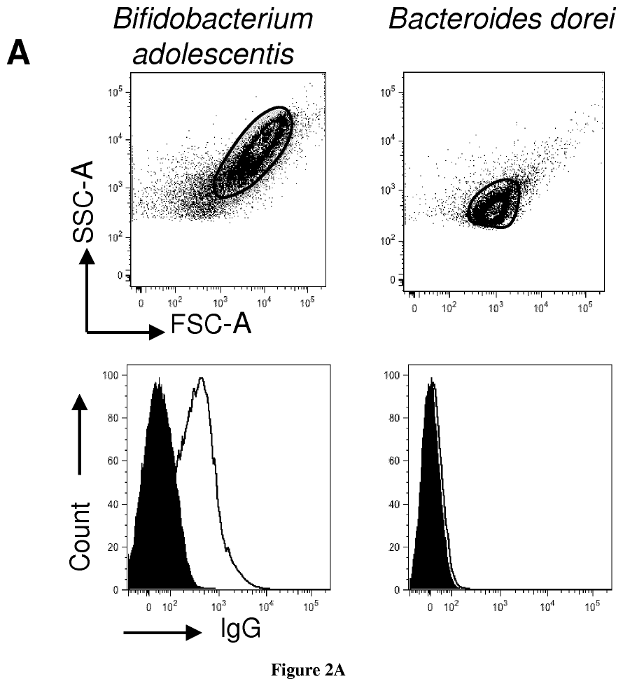

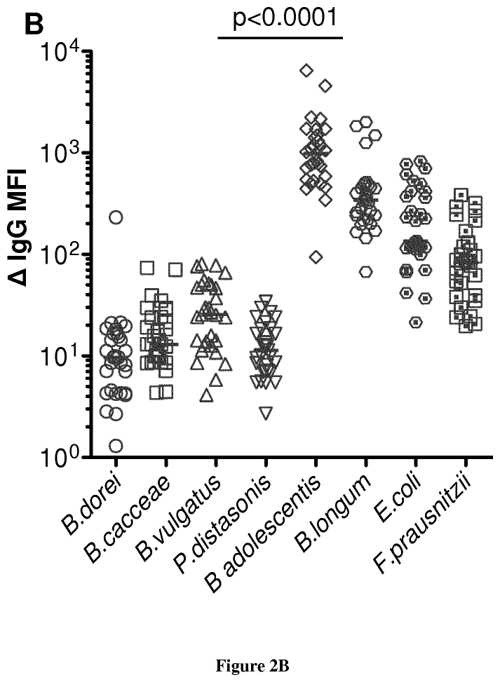

To confirm that systemic IgG binding is directed against IgA-bound bacteria, we evaluated in vitro serum IgG binding to cultivable bacterial strains. We selected four bacterial strains that were not preferentially bound by IgA in human feces and four others that were previously defined as classical IgA targets in vivo¹²⁻¹⁴. As shown in FIG. 2, IgG from healthy individuals (n=30) bind much more significantly Bifidobacterium longum, Bifidobacterium adolescentis, Faecalibacterium prausnitzii and Escherichia coli, known to be particularly enriched in the IgA-coated fraction of healthy individuals, than three different strains of Bacteroides sp. and Parabacteroides distasonis, known to be particularly enriched in the IgA-uncoated fraction of the fecal microbiota (FIG. 2A-B). The majority of anti-commensal IgG antibodies are of the IgG2b and IgG3 isotypes in mice. Using isotype-specific secondary antibodies we detected minimal IgG1 binding, but high seric IgG2 reactivity, to Bifidobacterium adolescentis, Bifidobacterium longum and Escherichia coli, suggesting that IgG2 is involved in commensals targetting in humans (FIG. S3).

Since anti-commensal IgG might possibly be triggered during mucosal immune responses, we characterized lamina propria B cells and detected the presence of IgG2+ B cells throughout the intestine (FIG. S4). Of note, IgG transcripts are more abundant in LP tissue that in PBMCs, as measured by qPCR (FIG. S4).

These results demonstrate that human IgG recognize a wide range of commensal under homeostatic conditions. Systemic humoral immunity (notably IgG2) converges with mucosal immunity to bind the surface of commensals.

2/Inter-Individual Variability and Non Overlapping Anti-Commensal IgA and IgG Molecular Targets.

It was previously suggested that murine IgG would target a restricted number of bacterial proteins and favored highly conserved outer membrane proteins⁸. Reactivity of human serum IgG against bacterial lysates from a Gram-negative strains was evaluated by immunoblotting. We observed that IgG labeled several E. coli bands (FIG. 2C), suggesting that multiple bacterial products are involved in the induction of systemic antibodies.

Interestingly, this analysis reveals a great deal of inter-individual variability, as it is not always the same bacterial products that react with the tested serums. We then compared the overlap between bacterial products labeled by IgG and IgA and found distinct binding profiles (FIG. 2C). Finally, in the 5 individuals tested, although some bacterial products (notably a 15 Kd antigen) are frequently targeted in most subjects and without isotype restriction, it clearly appears that IgA and IgG never share exactly the same binding pattern at a molecular level.

Taken together, these results demonstrate although IgG converges with IgA to bind the surface of commensals, it appears that IgA and IgG do not systematically target the same bacterial antigens, even at the individual level.

3/Private Anti-Microbiota IgG Specificities are Induced in IgA-Deficient Patients

The existence of seric IgG able to bind IgA-coated bacteria could equally suggest that some gut bacteria (or bacterial antigens) might cross the intestinal barrier: (i) in spite of IgA, or (ii) because of IgA. In order to explore these two putatively opposing roles for IgA, we studied the systemic anti-commensal IgG response in SIgAd. These patients had undetectable seric and digestive IgA levels while seric IgG were in the normal range¹⁵. Anti-microbiota IgG levels were significantly higher in SIgAd compared to controls (median [min-max]%;

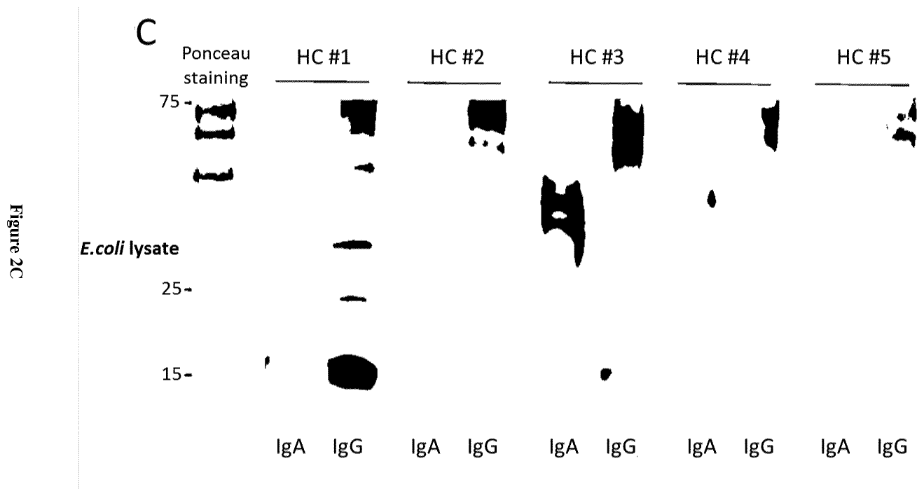

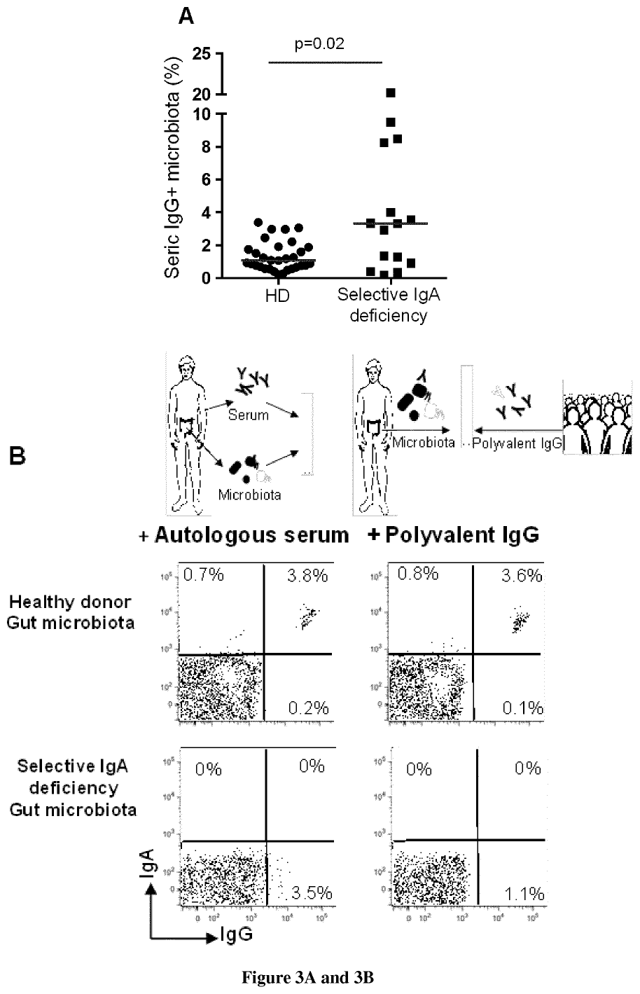

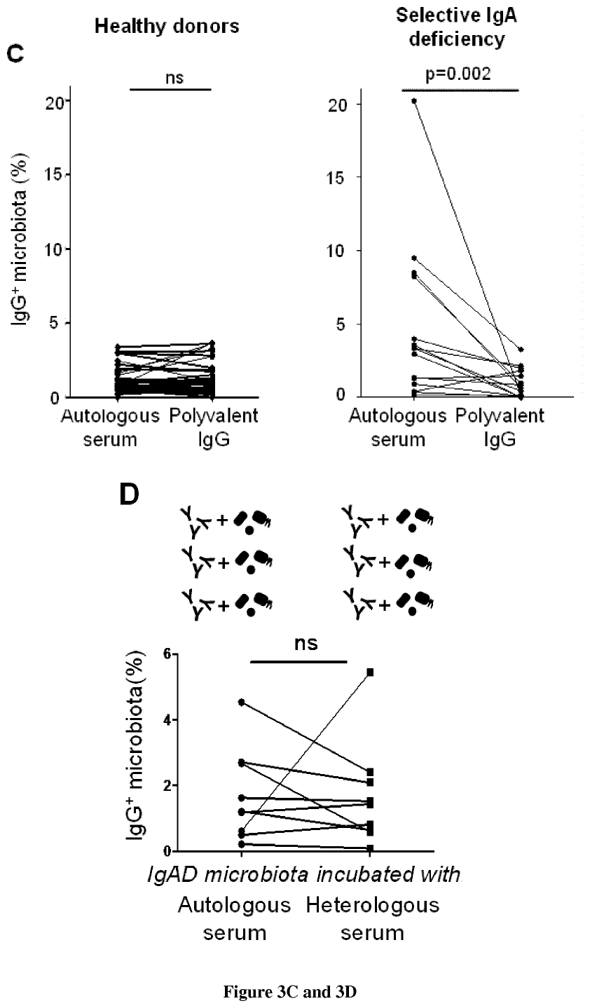

3.3[0.2-20.2]% versus 1.1%[0.2-3.2]%; FIG. 3A). Using irrelevant human IgG, we confirmed that, like in healthy donors, IgG interact with fecal bacteria in a Fab-dependent manner (Figure S2B). These data support an enhanced triggering of systemic IgG immunity against fecal microbiota when lacking secretory IgA, as shown in the murine model of polymeric immunoglobulin receptor deficiency⁶.

Considering this high level of anti-microbiota IgG in SIgAd, and the similarity of SIgAd and healthy microbiota composition¹⁵, we investigated how anti-microbiota IgG repertoires from healthy donors and IgA deficient patients were overlapping. Using polyclonal IgG from pooled serum of healthy donors, we assessed IgG-bound microbiota using either healthy or SIgAd purified microbiota. We showed that pooled polyclonal IgG and autologous healthy sera recognized a similar percentage of fecal bacteria (median [min-max]%;1[0-3.7] % vs 1.1[0.2-3.2]%, respectively, FIG. 3B-C). In contrast, pooled polyclonal IgG bound a smaller bacterial fraction of IgA deficient-microbiota compared to autologous patient serum (median [min-max]%;0.4[0-3.6] % vs 3.3[0.2-20.2] %, FIG. 3B-C). In order to test whether similar specificities are induced in all or most IgA deficient individuals, we compared their IgG reactivity to autologous or heterologous gut microbiota. In this experiment (FIG. 3D), each IgA-deficient microbiota was incubated either with autologous serum (i.e.: autologous condition), or with serum from an unrelated IgA deficient individual (i.e.: heterologous condition). As shown in FIG. 3D, no significant difference was seen between autologous or heterologous conditions (median autologous IgG+ microbiota 1.2% versus median heterologous IgG+ microbiota 1.4%). Of note, heterologous seric IgG also predominantly interact with fecal microbiota in a Fab-dependent manner (FIG. S2C).

This set of data suggests that peculiar anti-microbiota IgG specificities are induced in IgA-deficient patients, but not in healthy individuals.

4/IgG Specifically Recognize a Broad Spectrum of Bacteria

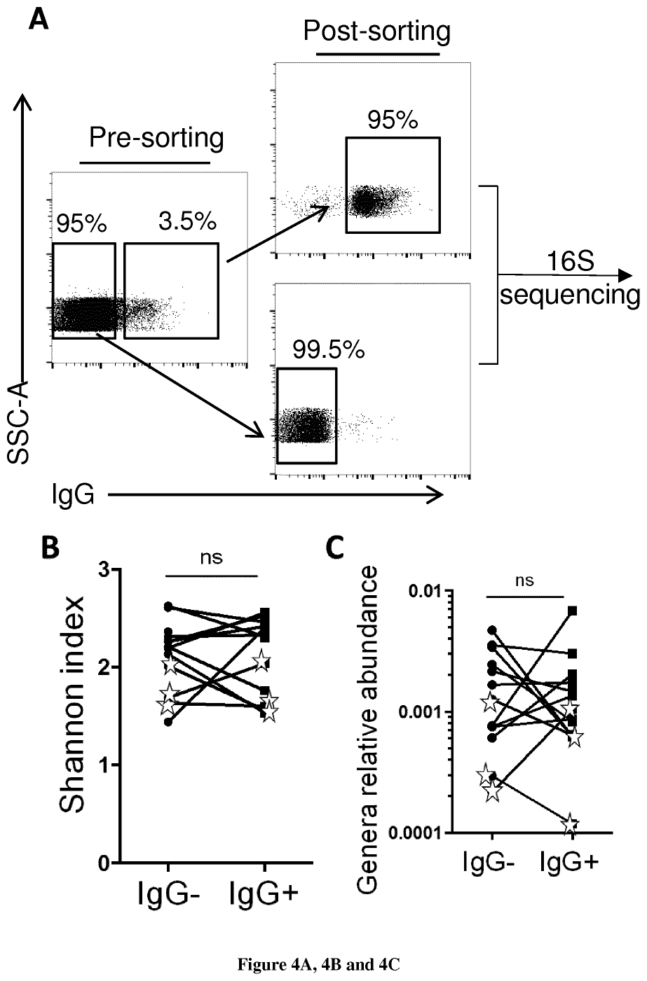

To more deeply decipher anti-commensal IgG specificities in both healthy donors and IgA deficient patients, we next performed a stringent flow-sorting to isolate IgG-bound bacteria and identified their taxonomy by 16S rRNA sequencing (FIG. 4A). We observed extensive inter-individual variability at genus level irrespective of immunological status (healthy donors vs IgA deficient patients). Microbial diversity calculated by Shannon index varied between donors, but on average bacterial diversity of IgG⁺ and IgG⁻ bacteria was not significantly different (FIG. 4B). We postulated that IgG might preferentially interact with dominant taxa, and therefore compared relative abundance of IgG-bound and IgG-unbound genera. Both fractions exhibited equal distributions of rare and abundant genera (FIG. 4C), thus IgG target commensals irrespectively of their frequency. Interestingly, we found that individual IgG⁺ and IgG⁻ fecal bacterial profiles were remarkably different, supporting a strong IgG bias against peculiar taxa that cannot be explained by an expansion of the latter. Besides, anti-commensals IgG were not restricted to pathobionts, but also targeted symbiotic genera such as Faecalibacterium, whose the most common species (i.e.: F.prausnitzii) has been assigned anti-inflammatory properties in both healthy donors and IgA deficient patients¹⁶. From this part we conclude that anti-commensal IgG recognize a diverse array of both pathobionts and commensal bacteria. Importantly, each individual harbored a private IgG antimicrobial signature.

5/High Anti-Microbiota IgG Levels Correlate with Reduced Systemic Inflammation

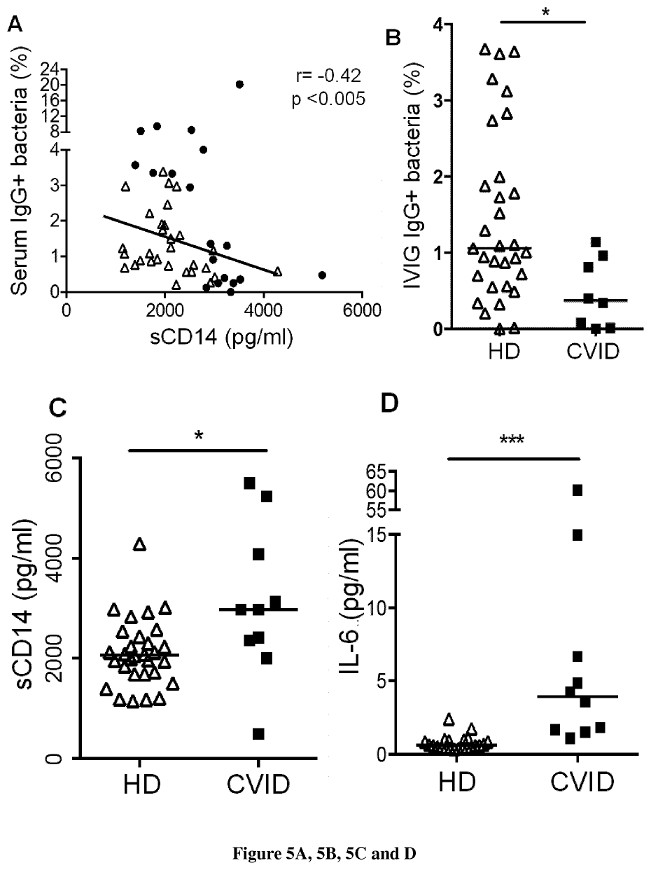

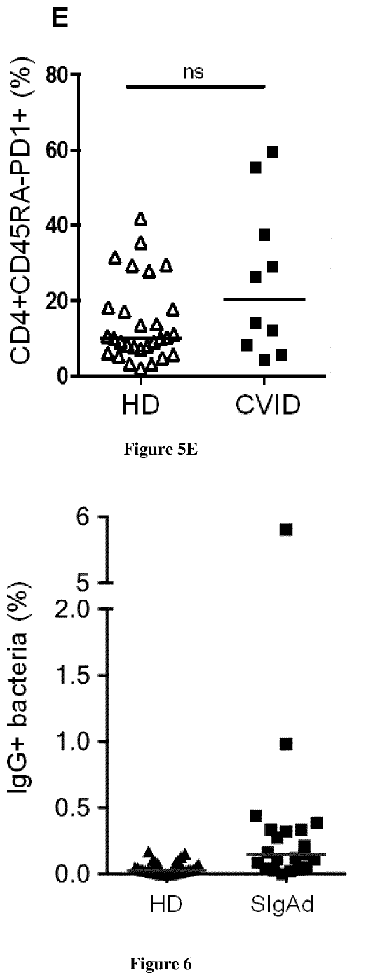

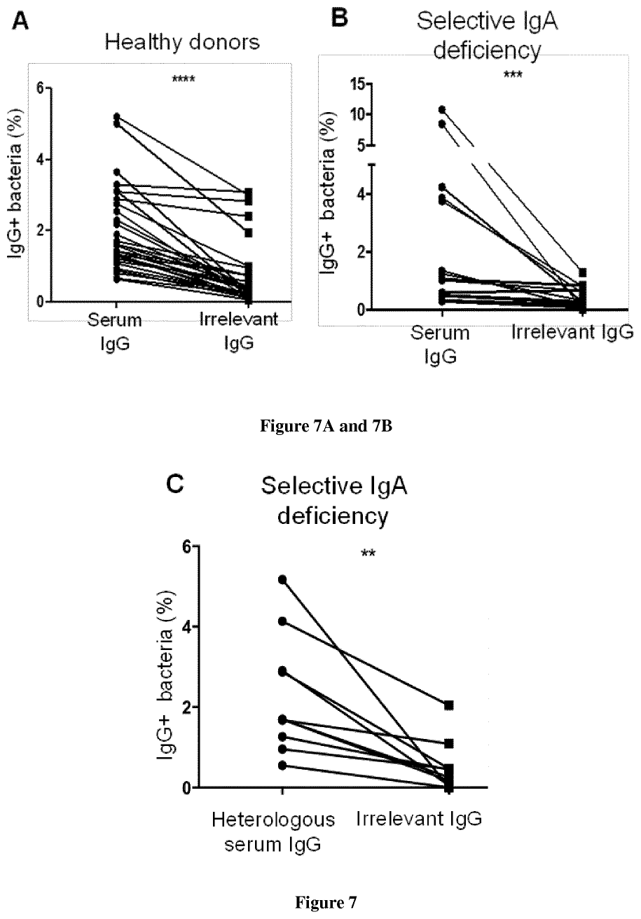

Microbiota-specific serum IgG responses contribute to symbiotic bacteria clearance in periphery and maintain mutualism in mice². We thus hypothesized that anti-commensals IgG might influence the balance of systemic inflammatory versus regulatory responses in humans. Hence, we measured plasma levels of sCD14 (a marker of monocyte activation,¹⁷) and observed that seric IgG-coated bacteria inversely correlated with soluble CD14 (r=-0.42, p<0.005; FIG. 5A) in both healthy donors and SIgAd patients. These results are in line with the finding that IgG replacement therapy reduced endotoxemia¹⁸. To further explore the potential link between anti-microbiota IgG and systemic inflammation, we explored CVID patients (characterized by both IgG and IgA defects). These patients benefit from IVIG treatment. Yet, we show that IVIG do not efficiently bind CVID microbiota. As shown in FIG. 5B, IVIG bound a reduced fraction of CVID microbiota compared to control microbiota (median [min-max]%; 0.37[0.00-1.14]% vs 1.06[0.00-3.7]%). We then determined plasma levels of sCD14 and IL-6 (an inflammatory cytokine reflecting T-cell activation) and evaluated the expression of PD-1 (a T-cell co-inhibitory molecule induced after activation) on CD4+ T cells. IL-6 as well as sCD14 levels were consistently higher in CVID patients than in healthy donors (IL-6, median [min-max]%, 1.8(0.7-60.1) pg/ml versus 0.6(0.33-2.4) pg/ml; sCD14, median [min-max]%; 2063 (590-5493) pg/ml versus median 2696(1147-4283) pg/ml; FIG. 5C-D). Moreover, CD45RA-PD1+CD4+T cells tended to increase in CVID patients, as compared with healthy donors (median [min-max]%; 20.3(4.26-59.6)% versus 10(2.09-41.9)%, FIG. 5E).

Altogether, in both controls and IgA-deficient patients, systemic anti-microbiota IgG responses correlate with reduced inflammation.

Discussion

Anti-commensal IgG have been described in patients with inflammatory diseases ^{5,19,20}. Here, we characterize for the first time a broad anti-commensal IgG response under homeostatic conditions in humans. Previous work demonstrated that symbiotic Gram-negative bacteria disseminate spontaneously and drive systemic IgG responses⁸. We show here that a diverse array of commensal bacteria, including Gram-positive and Gram-negative species, can induce systemic IgG. We show that a pathobiont like E. coli induce less systemic IgG responses than a presumably beneficial symbiont like B. adolescentis (FIG. 2B). Therefore the systemic IgG response in healthy humans does not appear preferentially driven by pathobionts, but also by commensals. In mice it has been shown that commensal microbes induce serum IgA responses that protect against sepsis²¹, illustrating the consequence of systemic anti-microbial IgA binding to both pathogenic strains and commensals. We postulate that systemic anti-microbiota IgG, also mainly induced by commensals, could have the same protective role.

Strikingly, systemic IgG and secretory IgA converge towards the same autologous microbiota subset. Yet, it seems unlikely that secretory IgA enhances systemic IgG responses, since IgA deficiency is associated with high proportions of IgG+ microbiota, as detected using bacterial flow cytometry on SIgAd microbiota labeled with autologous serum. In addition, induction of anti-commensal IgG has been shown to be microbiota-dependent, but IgA-independent in mice^{2,6}. Systemic IgG could reflect asymptomatic gut microbiota translocation episodes in healthy individuals. Repeated bacterial translocations might occur more frequently in the absence of secretory IgA, accounting for elevated anti-microbiota IgG levels in these patients.

IgA do not activate complement via the classical pathway²². Interestingly, the anti-Bifidobacterium adolescentis IgG response is primarily restricted to the IgG2 isotype (FIG. S3), which less efficiently triggers the classical route of complement than IgG1 and IgG3²³. Furthermore, IgG2 poorly interact with type I FcγRs, while IgG1 and IgG3 demonstrate affinity for most FcγRs²⁴. These distinct binding patterns have functional consequences. IgG1 antibodies mediate phagocytosis and induce potent pro-inflammatory pathways while IgG2 are rather involved in dendritic cell or B cell activation^{25,26}. Besides its specific Fc domain interaction, IgG2 is usually, but not exclusively, associated with anti-carbohydrate responses²⁷. IgA was also recently shown to bind multiple microbial glycans²⁸. Thus, IgA and IgG2 could be viewed as playing similar roles, but in different compartments. Much effort has been recently expended to develop bacterial glycan or protein microarray. Glycomics could represent a new option in order to better decipher anti-microbiota antibody targets^{27,29}.

Importantly, we show that IgA and IgG do not systematically target the same bacterial antigens at an individual level (FIG. 2C). Therefore IgG and IgA epitopes are not strictly overlapping. This result could further illustrate antibacterial IgA/IgG synergy, and explain the absence of isotype competition allowing the observed IgA/IgG co-staining of bacteria (FIG. 1).

Recent studies suggested that murine secretory IgA are polyreactive and bind a broad but defined subset of microbiota^{30,31}. Similarly, up to 25% of intestinal IgG⁺ plasmablasts could produce polyreactive antibodies⁹. We therefore hypothesized that the cross-reactive potential of anti-commensal IgG may act as a first line of defense against potentially harmful bacteria. In line with this idea, it can be noted that homeostatic anti-commensal IgG confer protection against pathogens such as Salmonella⁸. Conversely, IgG directed against Klebsiella pneumoniae, an opportunistic pathogen, cross-react with commensal microbes³². Clonally related memory B cells expressing cross-specific anti-K. pneumoniae antibodies were found in both lamina propria and peripheral blood in humans suggesting that generation of anti-commensal antibodies might be triggered in the mucosal compartment. At the same time, anti-commensal memory B cells might recirculate in periphery³². Altogether, it appears possible that bacteria-specific IgG would arise from the gut, as all bacteria-specific IgG isotypes we characterized in human sera are also present in the gut (FIG. S4), and also because a large proportion of gut IgG+ B cells are expected to be commensal-specific⁹. However, it remains presently unknown whether serum IgG responses mainly originate from the gut and/or are induced the periphery following bacterial translocation.

We report that each individual harbors a private set of anti-commensal IgG in both healthy donors and IgA deficient patients. Since our analysis was limited to 3 IgA deficient patients, further study might precisely reveal how SIgAd anti-commensal IgG bind a distinct set of commensals. While IVIG preparations contain an extended set of anti-commensal IgG, we observe that IVIG less efficiently bind CVID microbiota. These observations are consistent with reported alterations of gut microbiota in CVID patients³³. Microbiota perturbations are also associated with selective IgA deficiency. The latter perturbations are less pronounced than in CVID, since the presence of IgM appears to preserve SIgAd microbiota diversity¹⁵. Nevertheless, IgA deficiency condition is also associated in severe cases with bacterial translocation, colitis and dysbiosis. These complications are not accessible to substitutive Ig replacement therapy³⁴. Indeed, IVIG do not appear to contain high-enough concentrations as well as appropriate specificities of anti-commensal IgG. As shown in FIG. 3, healthy control serum usually less efficiently binds IgA deficient microbiota than autologous serum. Similarly, IVIG poorly targets CVID gut microbiota (FIG. 5B). In addition, local mucosal antibody responses might be important in regulating microbiota composition in a way that cannot be substituted by IVIG. These findings expand our understanding of how IVIG fail to treat gastro-intestinal symptoms in CVID and IgA deficient patients. Dysbiosis and gastro-intestinal complications might not accessible to substitutive Ig replacement therapy, since, as we show, healthy IgG repertoire does not contain adequate “dysbiotic-specific” antibodies.

It was recently shown in mice that maternally-derived anti-commensal IgG dampen aberrant mucosal immune responses and strengthen epithelial barrier^{7,35}. The contribution of systemic anti-commensal IgG to the regulation of microbiota/immune homeostasis was not explored in the latter studies. Here, we show that anti-commensal IgG are negatively associated with sCD14, suggesting they might quell inflammation. In support of this, we measured higher levels of sCD14 and IL-6 in plasma of patients lacking both IgA and IgG compared to controls (FIG. 5).

Altogether, these data suggest that systemic IgG and intestinal IgA cooperate in different body compartments to limit systemic pro-inflammatory pathways. While selective IgA deficient patients harbour elevated seric anti commensal IgG levels, CVID patients can not mount an appropriate IgG response. These findings suggest that : in selective IgA deficiency, microbiota confinement is obtained at the price of a strong inflammatory response, and in CVID, confinement is lost and Ig replacement therapy do not substitute for a specific autologuous IgG response. We therefore propose that IgA supplementation might have beneficial effects on gut dysbiosis and systemic inflammatory disorders associated with antibody deficiencies. IgA might be orally delivered through a carrier system allowing colon delivery. Polymers such as gellan gum or pectin, are degraded specifically by the colonic microbiota and could thus release polymer-bound IgA locally³⁶.

In summary, we report for the first time a systemic anti-commensal IgG response that is restricted to intestinal IgA-coated bacteria in humans. We demonstrate that in the absence of IgA, anti-commensal IgG responses are amplified and associated with reduced systemic inflammation. Finally, the present study provides new therapeutic perspectives based on IgA supplementation in patients with CVID or SIgAd, while SIgAd -derived IgG supplementation might be considered in CVID.

### REFERENCES

Throughout this application, various references describe the state of the art to which this invention pertains. The disclosures of these references are hereby incorporated by reference into the present disclosure.

1. Honda K, Littman D R. The microbiota in adaptive immune homeostasis and disease. Nature. 2016;535:75.

2. Slack E, Hapfelmeier S, Stecher B, Velykoredko Y, Stoel M, Lawson M A E, et al. Innate and adaptive immunity cooperate flexibly to maintain host-microbiota mutualism. Science. 2009;325:617-20.

3. Donskey C J. The role of the intestinal tract as a reservoir and source for transmission of nosocomial pathogens. Clin Infect Dis Off Publ Infect Dis Soc Am. 2004;39:219-26.

4. MacFie J. Current status of bacterial translocation as a cause of surgical sepsis. Br Med Bull. 2004;71:1-11.

5. Beaugerie L, Sokol H. Clinical, serological and genetic predictors of inflammatory bowel disease course. World J Gastroenterol. 2012;18:3806-13.

6. Johansen F E, Pekna M, Norderhaug IN, Haneberg B, Hietala M A, Krajci P, et al. Absence of epithelial immunoglobulin A transport, with increased mucosal leakiness, in polymeric immunoglobulin receptor/secretory component-deficient mice. J Exp Med. 1999;190:915-22.

7. Koch M A, Reiner G L, Lugo K A, Kreuk L S M, Stanbery A G, Ansaldo E, et al. Maternal IgG and IgA Antibodies Dampen Mucosal T Helper Cell Responses in Early Life. Cell. 2016;165:827-41.

8. Zeng M Y, Cisalpino D, Varadarajan S, Hellman J, Warren H S, Cascalho M, et al. Gut Microbiota-Induced Immunoglobulin G Controls Systemic Infection by Symbiotic Bacteria and Pathogens. Immunity. 2016;44:647-58.

9. Benckert J, Schmolka N, Kreschel C, Zoller M J, Sturm A, Wiedenmann B, et al. The majority of intestinal IgA+ and IgG+ plasmablasts in the human gut are antigen-specific. J Clin Invest. 2011;121:1946-55.

10. Iversen R, Snir O, Stensland M, Kroll J E, Steinsbo Ø, Korponay-Szabó I R, et al. Strong Clonal Relatedness between Serum and Gut IgA despite Different Plasma Cell Origins. Cell Rep. 2017;20:2357-67.

11. Moor K, Fadlallah J, Toska A, Sterlin D, Balmer M L, Macpherson A J, et al. Analysis of bacterial-surface-specific antibodies in body fluids using bacterial flow cytometry. Nat Protoc. 2016;11:1531-53.

12. Palm N W, de Zoete M R, Cullen T W, Barry N A, Stefanowski J, Hao L, et al. Immunoglobulin A coating identifies colitogenic bacteria in inflammatory bowel disease. Cell. 2014;158:1000-10.

13. D′Auria G, Peris-Bondia F, D\hat{z}nková M, Mira A, Collado M C, Latorre A, et al. Active and secreted IgA-coated bacterial fractions from the human gut reveal an under-represented microbiota core. Sci Rep. 2013;3:3515.

14. Kau AL, Planer J D, Liu J, Rao S, Yatsunenko T, Trehan I, et al. Functional characterization of IgA-targeted bacterial taxa from undernourished Malawian children that produce diet-dependent enteropathy. Sci Transl Med. 2015;7:276ra24.

15. Fadlallah J, El Kafsi H, Sterlin D, Juste C, Parizot C, Dorgham K, et al. Microbial ecology perturbation in human IgA deficiency. Sci Transl Med. 2018;10.

16. Sokol H, Pigneur B, Watterlot L, Lakhdari O, Bermúdez-Humarán L G, Gratadoux J-J, et al. Faecalibacterium prausnitzii is an anti-inflammatory commensal bacterium identified by gut microbiota analysis of Crohn disease patients. Proc Natl Acad Sci USA. 2008;105:16731-6.

17. Bazil V, Strominger JL. Shedding as a mechanism of down-modulation of CD14 on stimulated human monocytes. J Immunol Baltim Md 1950. 1991;147:1567-74.

18. Perreau M, Vigano S, Bellanger F, Pellaton C, Buss G, Comte D, et al. Exhaustion of bacteria-specific CD4 T cells and microbial translocation in common variable immunodeficiency disorders. J Exp Med. 2014;211:2033-45.

19. Landers C J, Cohavy O, Misra R, Yang H, Lin Y-C, Braun J, et al. Selected loss of tolerance evidenced by Crohn's disease-associated immune responses to auto- and microbial antigens. Gastroenterology. 2002;123:689-99.

20. Macpherson A, Khoo U Y, Forgacs I, Philpott-Howard J, Bjarnason I. Mucosal antibodies in inflammatory bowel disease are directed against intestinal bacteria. Gut. 1996;38:365-75.

21. Wilmore J R, Gaudette B T, Gomez Atria D, Hashemi T, Jones D D, Gardner C A, et al. Commensal Microbes Induce Serum IgA Responses that Protect against Polymicrobial Sepsis. Cell Host Microbe. 2018;23:302-311.e3.

22. Russell M W, Mansa B. Complement-fixing properties of human IgA antibodies. Alternative pathway complement activation by plastic-bound, but not specific antigen-bound, IgA. Scand J Immunol. 1989;30:175-83.

23. Bindon C I, Hale G, Brüggemann M, Waldmann H. Human monoclonal IgG isotypes differ in complement activating function at the level of C4 as well as C1q. J Exp Med. 1988;168:127-42.

24. Bruhns P, Iannascoli B, England P, Mancardi D A, Fernandez N, Jorieux S, et al. Specificity and affinity of human Fcgamma receptors and their polymorphic variants for human IgG subclasses. Blood. 2009;113:3716-25.

25. Nimmerjahn F, Gordan S, Lux A. FcγR dependent mechanisms of cytotoxic, agonistic, and neutralizing antibody activities. Trends Immunol. 2015;36:325-36.

26. White A L, Chan H T C, French R R, Willoughby J, Mockridge C I, Roghanian A, et al. Conformation of the human immunoglobulin G2 hinge imparts superagonistic properties to immunostimulatory anticancer antibodies. Cancer Cell. 2015;27:138-48.

27. Schneider C, Smith D F, Cummings R D, Boligan K F, Hamilton R G, Bochner B S, et al. The human IgG anti-carbohydrate repertoire exhibits a universal architecture and contains specificity for microbial attachment sites. Sci Transl Med. 2015;7:269ra1.

28. Bunker J J, Erickson S A, Flynn T M, Henry C, Koval J C, Meisel M, et al. Natural polyreactive IgA antibodies coat the intestinal microbiota. Science. 2017;

29. Christmann B S, Abrahamsson T R, Bernstein C N, Duck L W, Mannon P J, Berg G, et al. Human seroreactivity to gut microbiota antigens. J Allergy Clin Immunol. 2015;136:1378-1386-5.

30. Bunker J J, Flynn T M, Koval J C, Shaw D G, Meisel M, McDonald B D, et al.

Innate and Adaptive Humoral Responses Coat Distinct Commensal Bacteria with Immunoglobulin A. Immunity. 2015;43:541-53.

31. Okai S, Usui F, Yokota S, Hori-i Y, Hasegawa M, Nakamura T, et al. High-affinity monoclonal IgA regulates gut microbiota and prevents colitis in mice. Nat Microbiol.

32. Rollenske T, Szijarto V, Lukasiewicz J, Guachalla LM, Stojkovic K, Hartl K, et al. Cross-specificity of protective human antibodies against Klebsiella pneumoniae LPS O-antigen. Nat Immunol. 2018;19:617-24.

33. Jørgensen S F, Troseid M, Kummen M, Anmarkrud J A, Michelsen A E, Osnes L T, et al. Altered gut microbiota profile in common variable immunodeficiency associates with levels of lipopolysaccharide and markers of systemic immune activation. Mucosal Immunol. 2016;9:1455-65.

34. Favre O, Leimgruber A, Nicole A, Spertini F. Intravenous immunoglobulin replacement prevents severe and lower respiratory tract infections, but not upper respiratory tract and non-respiratory infections in common variable immune deficiency. Allergy. 2005;60:385-90.

35. Gomez de Agiiero M, Ganal-Vonarburg S C, Fuhrer T, Rupp S, Uchimura Y, Li H, et al. The maternal microbiota drives early postnatal innate immune development. Science. 2016;351:1296-302.

36. Sandolo C, Péchiné S, Le Monnier A, Hoys S, Janoir C, Coviello T, et al. Encapsulation of Cwp84 into pectin beads for oral vaccination against Clostridium difficile. Eur J Pharm Biopharm Off J Arbeitsgemeinschaft Pharm Verfahrenstechnik EV. 2011;79:566-73.

37. Juste C, Kreil D P, Beauvallet C, Guillot A, Vaca S, Carapito C, et al. Bacterial protein signals are associated with Crohn's disease. Gut. 2014;63:1566-77.

38. Caporaso J G, Kuczynski J, Stombaugh J, Bittinger K, Bushman F D, Costello EK, et al. QIIME allows analysis of high-throughput community sequencing data. Nat Methods. 2010;7:335-6.

39. Cole J R, Wang Q, Cardenas E, Fish J, Chai B, Farris R J, et al. The Ribosomal Database Project: improved alignments and new tools for rRNA analysis. Nucleic Acids Res. 2009;37:D141-145.

1. A composition of IVIGs (Intravenous immunoglobulins) containing at least 1% of immunoglobulin G (IgG) from SIgAd (Selective IgA deficiency) patients.

2. The composition of IVIGs according to claim 1, wherein said composition contains between 1% to 10% of immunoglobulin G (IgG) from SIgAd (Selective IgA deficiency) patients.

3. A method of preparation of the composition of IVIGs according to claim 1 comprising
separating plasma proteins into individual stable fractions with different biological functions by Cohn's fractionation; or
purifying immunoglobulins by ion-exchange chromatography.

4. A method of treating an antibody deficiency disorder selected from the group consisting of an immune disease, an inflammatory disorder and an autoimmune disease, in a subject in need thereof, comprising
administering to the subject a therapeutically effective amount of a pharmaceutical composition comprising the composition of IVIGs of claim 1.

5. The method according to claim 4, wherein the immune disease is a Primary antibody deficiency or a Secondary antibody deficiency.

6. The method according to claim 5 wherein the Primary antibody deficiency is common variable immunodeficiency (CVID).

7. The method according to claim 4, wherein the inflammatory disorder is selected form the group consisting of a gut inflammatory disease, sepsis and graft versus host disease.

8. The method according to claim 7 wherein the gut inflammatory disease is inflammatory bowel disease.

9. The method according to claim 4, wherein the autoimmune disease is a neurological, haematological, nephrological, rheumatological and/or dermatological disease.
